(19) United States
(12) Patent Application Publication
(10) Pub. No.: US 2004/0143145 A1
(43) Pub. Date: Jul. 22, 2004

(54) Formation of gas hydrates by fluidized bed granulation

(76) Inventors: Phillip D. Servio, Montreal (CA); Arturo M. Macchi, Montreal (CA); Luiz B. Da Silva, Danville, CA (US)

Correspondence Address: John P. Wooldridge, Esq., Suite 110, 535 Lipoa Parkway, Kihei, HI, 96753, US

(21) Appl. No.: 10/754,162

(22) Filed: Jan. 7, 2004

Related U.S. Application Data
(60) Provisional application No. 60/438,571, filed on Jan. 7, 2003.

(51) Int. Cl. C07C 9/00

(52) U.S. Cl. 585/15

(57) ABSTRACT

A steady-state method for producing gas hydrates provides seed gas hydrate particles in a reaction chamber, flows a hydrate-forming gas into the reaction chamber and flows water into the reaction chamber to produce several possible reactions. One reaction occurs from the combination of the seed gas hydrate particles, the hydrate-forming gas and the water to provide gas hydrate growth onto the seed gas hydrate particles. Another reaction occurs from the interaction of the hydrate-forming gas and the water to form new gas hydrate particles. Material is removed from the reaction chamber and fragmented and some of fragmented gas hydrate particles are recycled back into the reaction chamber.

This application claim priority to U.S. Provisional Patent Application Serial No. 60/438,571, filed Jan. 7, 2003, titled: “Formation Of Gas Hydrates By Fluidized Bed Granulation,” incorporated herein by reference.

### BACKGROUND OF THE INVENTION

1. Field of the Invention

The present invention relates to gas hydrates, and more specifically, it relates to a process for the production of gas hydrate granules in a fluidized bed whereby water is contacted with gas hydrate particles and a gas or mixture of gases known to produce gas hydrates under proper thermodynamic conditions.

1. Description of Related Art

Potential benefits have been associated with the exploitation of gas hydrates. Gas hydrates are non-stoichiometric crystalline compounds that belong to the inclusion group known as Clathrates. Hydrates occur when water molecules attach themselves through hydrogen bonding and form cages that can be occupied by a single gas or volatile liquid molecule. The presence of a gas or volatile liquid inside the water network thermodynamically stabilizes the structure through physical bonding via weak van der Waals forces. Naturally occurring hydrates, containing mostly methane, exist in vast quantities within and below the permafrost zone and in sub-sea sediments and are being looked upon as a future energy source. At present, the amount of organic carbon entrapped in hydrate exceeds all other reserves (fossil fuels, soil, peat, and living organisms) (Seuss et al., 1999).

An important benefit of gas hydrates deals with the transportation and storage of natural gas. Khokhar et al. (1998) reported a study demonstrating that transport of natural gas from the northern North Sea to Central Europe in hydrated form compared to liquefied natural gas can reduce overall costs by 24%. FIG. 1 shows a comparison of three different methods to store and transport natural gas. Each method demonstrates the thermodynamic conditions and phases required for a 1 m³ container to store an equivalent amount of natural gas (160 m³ at STP) when expanded to standard temperature and pressure. Gas hydrates require the more cost effective storage conditions, but necessitate a safe and efficient method for their production.

Natural gas hydrates will also be important for the development of hydrogen, methanol and solid-oxide fuel cells since all three can directly use or convert methane to produce the desired fuel. Carbon dioxide hydrate is also an important hydrate. Carbon dioxide is a major contributor to global warming and, following the Kyoto protocol, several countries have set a carbon dioxide emissions target of 6% below the year 1990 levels by year 2008-2012. Work is being conducted on capturing carbon dioxide by transforming it into hydrates (Brewer, P G., Peltzer, E T., Friederich, G., Aya, I. and Yamane, K., Experiments on the Ocean Sequestration of Fossil Fuel CO₂: pH Measurements and Hydrate Formation, Marine Chemistry, 72 (2-4), 83-93, 2000).

Gudmundsson describes various systems for making gas hydrates (see U.S. Pat. No. 5,536,893 and WO Patent Publication No. 93/01153). In a typical system of Gudmundsson, natural gas is compressed, cooled and fed to a continuously stirred tank reactor vessel. Water from a suitable source is pumped through a cooler to form water/ice slurry that is introduced into the stirred tank. The tank is maintained under conditions appropriate to produce a gas hydrate (e.g., 50° F., 720 psig). The gas hydrate slurry produced in the tank is transported to a separator where water is removed. The separator includes a series of cyclones and a rotary drum filter. Finally, the purified hydrates are frozen to 5° F. in a freezer, from where the hydrates are transferred to a storage or transport device. It is important to note that this process utilizes water as the continuous phase. Other examples of patents that produce hydrate in reactors where water is the continuous phase are Hutchinson et al. (1945) in U.S. Pat. No. 2,375,559, U.S. Pat. No. 2,904,511 to Donath (1959), U.S. Pat. No. 3,514,274 to Cahn et al. (1970) and U.S. Pat. No. 6,350,928 to Waycuilis et al. (2002).

U.S. Pat. No. 6,180,843 of Heinemann et al. (2001) resembles a fluidized spray drying process employed in the drying industry for handling slurries. In their process, water is finely dispersed above a fluidized bed. Some of the injected water forms seed hydrate particles, while the rest coats already-formed particles surrounding the atomizing nozzle. These particles receive successive coats of water and may agglomerate with neighboring particles until they reach a sufficient size and fall by gravity to the bottom of the vessel. The lower section of the vessel has a smaller cross-section and the particles will remain in suspension, absorbing more gas before finally exiting by the bottom of the fluidized bed.

The process of Heinemann et al. does not require recycling of particles to the fluidized bed. They leave this as an option for start-up. Thus, in order to maintain a constant inventory of particles in the bed and ensure continuous steady operation, fresh nuclei particles must be created in the fluidized bed by either the water atomization process (i.e., injected water droplets produce gas hydrates particles, not only coat surrounding particles) or by particles continuously fragmenting due to intense mixing in the bed.

The Heinemann et al. (2001) process presents favorable hydrate formation kinetics and is easier to operate than reactors where water is the continuous phase. However, it will still be less efficient than a process where there is an attempt to contact all the feed liquid with particles in the fluidized bed. The reasons are as follows:

1. The rate of conversion of water to hydrate (i.e., kinetics) is much greater if there is a precursor such as a seed particle that is already a hydrate than for an isolated water droplet in a gas stream.

2. The overall particle surface area available for the liquid to spread may be greater or, at least, not lower. In the Heinemann et al. process, the volumetric concentration of particles surrounding the nozzle may not be as high as the bottom of the chamber in the fluidized bed.

3. The heat transfer rate will be greater since all the liquid will transfer heat by both convection with the gas and conduction with the particles. Furthermore, by forming a thin film around the particles, the resistance to heat transfer is smaller than for a liquid droplet of the same volume.

4. If hydrates nucleate on water droplets they will create a thin film of hydrates, on the interface, enveloping a volume of unconverted water. This thin film will act as a barrier to further conversion of enclosed water into hydrate. Hence, another benefit of coating a hydrate seed with water is that the thin water layer can more effectively interact with the surrounding gas to form hydrate. Increasing the water-gas interaction will result in a more efficient and faster hydrate growth.

### SUMMARY OF THE INVENTION

This invention relates to a process for the production of gas hydrate granules in a fluidized bed whereby water is contacted with gas hydrate particles and a gas or mixture of gases known to produce gas hydrates under proper thermodynamic conditions. This process will have superior heat, mass and kinetic rates than others presently available, thus resulting in a greater volumetric product yield.

In steady-state operation, water is atomized onto hydrate particles in a fluidized bed. Particles grow by successive coating of hydrates similar to a “granulation” process. In this case, particle growth is dictated not only by heat and mass transfer, but also by hydrate formation kinetics. Particles are continuously removed from the bottom of the chamber and then fragmented. If desired, the fragmented hydrate particles can be fluidized in a subsequent chamber by a hydrate forming gas in order to increase the gas content in the hydrate cages (i.e., similar to an “absorption” process). A portion of these fragmented hydrate particles is recycled to the granulation chamber as seed particles and the remainder is kept as a product Potential fine particles present in the hydrate forming gases exiting the fluidized beds can be removed by cyclones or other gas-solid separation devices and returned to the granulation chamber as seed particles. The un-reacted hydrate forming gas is compressed, cooled and recycled to the reactor. Under steady-state operation, the entire process may operate at temperatures between 255-320 K and pressures ranging from 100-50,000 kPa. Examples of hydrate-forming gases include methane, propane, ethane, carbon dioxide, and other natural gas components.

### BRIEF DESCRIPTION OF THE DRAWINGS

FIG. 1 compares natural gas storage conditions (adapted from Khokhar et al., 1998).

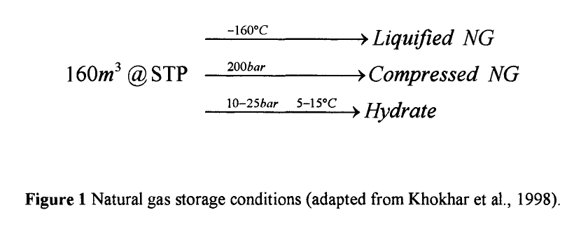

FIG. 2 is a schematic of the main components of the hydrate fluidized bed granulation process.

FIG. 3 is a schematic of the lower section of the hydrate fluidized bed granulation chamber

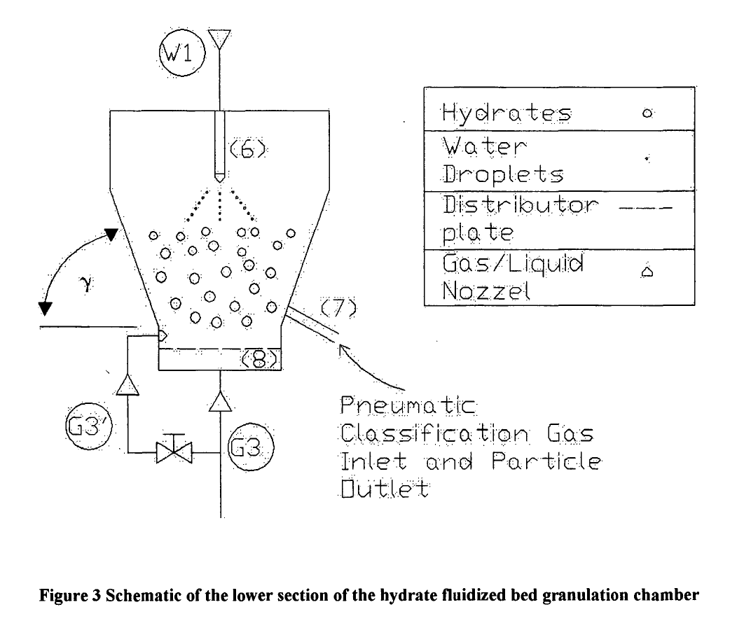

### DETAILED DESCRIPTION OF THE INVENTION

The invention utilizes a fluidized bed granulation process that allows the continuous production of gas hydrates. The principal advantages of this process are that it is simple, uses a minimal amount of equipment and is efficient, i.e., provides a large surface area for the hydrate reaction, has favorable heat and mass transfer rates and employs hydrates as seed particles. Hydrates are not likely to form quickly from water being atomized into a gas stream as with the Heinemann et al. (2001) and Gudmundsson (1996) processes due to the stochastic nature of hydrate crystal nucleation. On the other hand, water transforms into hydrates at a faster and predictable rate when contacted with hydrate seeds.

1. A start-up procedure is employed to create a bed of seed hydrate particles. It should be noted that the following procedure allows for the generation of hydrate seeds in situ. If the operator chooses to do so he may bypass the following by inserting hydrate crystals or any other suitable crystal seeds into the reactor. These seeds only serve as an aid to startup and at steady state hydrates generated within the process will be used as seeds.

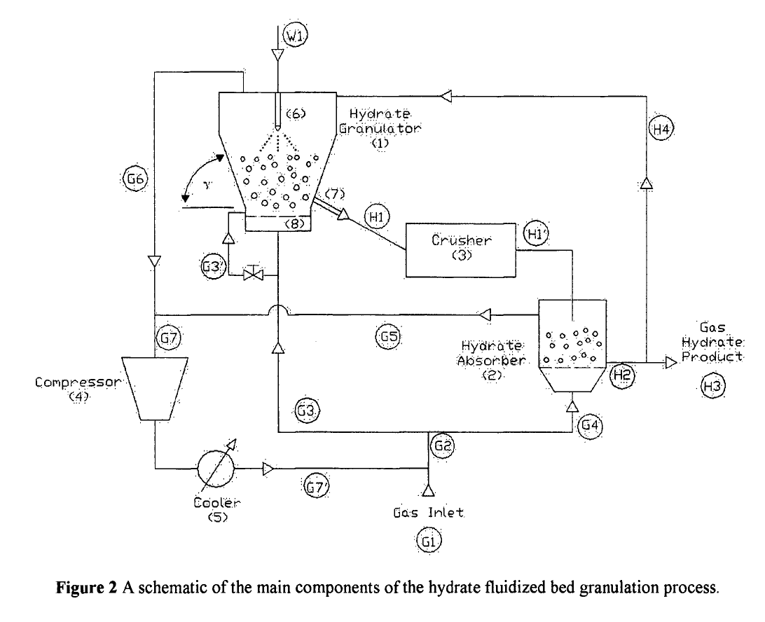

Referring to FIG. 2, at first, the temperature in the granulation chamber (1) is kept below the freezing point of water. Water (W1) is introduced from the top of the chamber (1) and contacted with a hydrate-forming gas (G3) in a countercurrent fashion in order to produce ice particles. Water is introduced by one or more atomizing devices (6) that provide the smallest possible droplet size and the highest possible surface to volume ratio, thus facilitating the nucleation of ice. The desired droplet size would be under 1000 micrometers. This step is similar to a spray drying process. A review of fluid atomizing devices is given by Masters, K, in “Spray Drying Handbook,” Longman Scientific and Technical, 1991.

These ice particles remain in suspension by keeping the gas flow rate (G3) above the point of minimum fluidization.

After sufficient ice granules have been formed, the water flow rate (W1) is reduced or even stopped and the temperature and pressure in the chamber (1) are increased to negate the possibility of ice crystals forming but sufficient to sustain hydrate growth, at least at the particle surface. The transition from ice to hydrate particles can be evaluated by monitoring pressure fluctuations (i.e., drop) in the chamber (1).

2. What follows is the description of the important constraints and features of the process operated at steady-state.

The number, geometry, locations (above and/or in the bed), positions (angle of fluid jets) and operating conditions (fluid flow rates and pressures) of the atomizing devices (6) are adjusted to provide optimal contact between the water droplets, gas and hydrate particles in the fluidized bed at the highest possible water throughput Optimal contact is achieved when all water droplets reach particles and these particles grow primarily by successive coating of hydrates (i.e., layering) rather than agglomeration of multiple hydrate particles. U.S. Pat. No. 6,159,252 of Schutte et al. (2000) presents several options for the locations and positions of fluid nozzles to achieve a high throughput of liquid during fluidized bed granulation operations.

The fluidized bed will primarily remain in the tapered section (angle (γ) between 0 and 90°) of the granulation chamber (1) in order to provide good mixing conditions. A circulatory and cyclic motion can further be imparted to the particles by designing the gas distributor (8) with a greater open area at its center. It has been shown that “overlap gill” or “nostril-like” gas outlets in the distributor plate promote particle movement, thus reducing dead zones and the risk of particles clogging the gas distributor (U.S. Pat. No. 6,159,252). Through these outlets, particles are obliquely fluidized at angles less than 90° relative to horizontal. One can also not use the gas distributor plate (8), thus avoiding potential clogging, and introduce the gas horizontally above the base of the granulation chamber through several nozzles (G3′).

Since the formation of hydrates is an exothermic process, the temperature in the granulation chamber (1) is continuously monitored and controlled between 255 and 320 K by adjusting the inlet temperature of the hydrate-forming gas (G3) and water (W1) streams with refrigeration units.

The pressure in the granulation chamber (1) is monitored and controlled between 100 and 50,000 kPa by adjusting the inlet pressures of the gas (G3), liquid (W1) and solid (H4) streams.

Since gas is consumed by the hydrate reaction and the average particle size, shape and density in the bed may fluctuate throughout the granulation operation, the gas volumetric flow rate (G3) is controlled to maintain smooth fluidization conditions and the bed height at an operating level.

The bed inventory is regulated by removing granulated hydrate particles (H1) and adding seed hydrate particles (H4), which are smaller in size. It is important to mention that the locations and operating conditions of the atomizing devices (6) and feed gas nozzles (G3′) may also contribute to generating seed particles in-situ by fragmenting the larger particles present in the bed, as described in U.S. Pat. No. 6,159,252 of Schutte et al. (2000), incorporated herein by reference. If it is possible to easily control the quantity and resulting size of the fragmented particles, this would be the preferred method of continuous hydrate seed generation over the use of an external embodiment such as the particle crusher (3).

Hydrate particles are discharged through one or several standpipes (7) placed near the bottom of the chamber (1) where there is a greater probability of removing particles larger is size than the bed average. These standpipes (7) can be located on the chamber side walls (side outlet) or on the gas distributor plate (bottom outlet). Furthermore, gas can be introduced in the particle discharge standpipe (7) in a countercurrent fashion to the particles for pneumatic classification. This will further increase the probability of removing the larger particles in the bed. There are several other classification devices (see Perry, R. H. and Green, D. W., Perry's Chemical Engineer's Handbook, McGraw-Hill, 1999) that can be implemented to the present process where the oversize discharged particles are fragmented, combined with undersized particles and then recycled back to the granulation unit, while the desired size particles are kept as final product or further processed in the fluidized bed absorber (2).

The removed hydrate particles (H1) from the granulation chamber (1) are then fragmented in a particle size reduction device (3). such as a crusher or roll mill as described by Rhodes, M. J., Principles of Powder Technology, Wiley, 1990 and Perry and Green (1999), incorporated herein by reference.

A portion (H4) of these fragmented hydrate particles is recycled to the granulation chamber (1) as seed particles. Optimally, these seeds should be introduced in the vicinity of the atomizing devices (6) situated above the fluidized bed.

The non-recycled portion (H3) of fragmented hydrates is kept as a product. In a final stage, the hydrates are compressed and stored in containers suitable for transport by truck, rail and/or sea.

If necessary, the fragmented hydrate particles (H1′) can be fluidized in a subsequent unit (2) by a hydrate-forming gas (G4) in order to fill or partially fill the remaining cages in the hydrate and to convert the free-water that may be present This is similar to an “absorption” process. The fluidized bed (2) can be a single chamber where the particle flow pattern is considered perfectly mixed. However, in order to obtain a tighter particle residence time distribution and thus a better product uniformity, the fluidized bed (2) may be staged (i.e., multiple chambers) where the particles flow in a crosscurrent or countercurrent manner to the hydrate forming gas. Although a countercurrent design may be more gas efficient for a single pass, the crosscurrent flow design is simpler to operate. Details of the design of multistage fluidized bed absorbers can be found in Kunii, D. and Levenspiel, O., Fluidization engineering, Butterworths, 1991, incorporated herein by reference.

The unconsumed gas streams (G5 and G6) exiting both fluidized beds are combined (G7) and then compressed, cooled and recycled. If necessary, a cyclone or other methods to remove particulates will be employed to remove potential fine particles generated in the fluidized beds. These fine hydrate particles would then be recycled to the granulation chamber (1) as seeds. Alternatively, the fines can be captured in-situ and returned to the respective fluidized beds by having the cyclones in the fluidized bed chambers.

As shown in FIG. 2, the main components of this process are a fluidized bed granulation unit (1), a particle size reduction unit (3) and possibly a fluidized bed absorption unit (2). The words granulation and absorption are used throughout the text with the understanding that these physical phenomena also include hydrate reactions.

The particle size reduction unit (e.g., crusher or roll mill) can be of standard design as described by Rhodes (1990) and Perry and Green (1999).

One embodiment for the fluidized bed granulator is a vessel that stands vertical. The top piece has a constant cross-section, while the bottom piece is tapered with an angle (γ) between 0 and 90°. Particles rest in the tapered section in order to give increased mixing conditions. Hydrate forming gas enters from the bottom of the bed, while the liquid may be injected from above and/or in the bed. Seed particles should preferably be introduced near the liquid injectors situated above the bed.

Another fluidized bed can be employed to further introduce gas into the fragmented hydrated particles. This fluidized bed can be a single chamber where the particle flow pattern is considered perfectly mixed or multiple chambers where the particle flow pattern can approach plug flow, see Kunii and Levenspiel (1991). One embodiment is a multi-stage fluidized bed with the particles flowing crosscurrent to the gas.

The fluidized beds can be constructed from metal (e.g., stainless steel 316, Platinum, Titanium, etc.) and have viewing ports made of transparent material such as Al₂O₃, PMMA, Polycarbonate, etc.

Finally, the process according to the invention may be performed in several known devices for fluidized bed granulation. One embodiment of the granulation chamber is tapered, but may be of other geometry, allowing the efficient contacting of a gas, solid and liquid in order to obtain optimal conditions for successive coating of the hydrate particles by a thin film of liquid at maximum liquid throughput There are several multiphase contacting modes that can be employed for this granulation process (see Geldart, 1986; Rhodes, 1990; Kunii and Levenspiel, 1991; Mujumdar, 1995; Fayed and Otten, 1997; Perry and Green, 1999; Yang, 2003). The variables affecting multiphase contacting may include the vessel geometry, the gas distributor design, the presence or absence of internals (e.g., draft-tube, heat exchangers), the physical properties of the phases, injection locations and operating conditions. The granulation process may operate as a fluidized, spouted or spout-fluid bed under various flow regimes to achieve the desired phase contact. Those skilled in the art of fluidized bed granulation will recognize that various changes and modifications can be made without departing from the spirit and scope of the invention, as defined in the appended claims.

The foregoing description of the invention has been presented for purposes of illustration and description and is not intended to be exhaustive or to limit the invention to the precise form disclosed. Many modifications and variations are possible in light of the above teaching. The embodiments disclosed were meant only to explain the principles of the invention and its practical application to thereby enable others skilled in the art to best use the invention in various embodiments and with various modifications suited to the particular use contemplated. The scope of the invention is to be defined by the following claims.

We claim:

1. A method for producing gas hydrates, comprising:
providing seed gas hydrate particles in a reaction chamber;
flowing a hydrate-forming gas into said reaction chamber;
flowing water into said reaction chamber, wherein at least one reaction occurs within said reaction chamber, wherein said at least one reaction is selected from the group consisting of (1) a first reaction wherein said seed gas hydrate particles, said hydrate-forming gas and said water react to provide gas hydrate growth onto said seed gas hydrate particles, wherein said seed gas hydrate particles having such growth are referred to herein as “growth particles,” and (2) a second reaction wherein said hydrate-forming gas and said water react to form new gas hydrate particles;
removing from said reaction chamber and fragmenting at least a portion of material selected from the group consisting of said growth particles, said new gas hydrate particles and said seed gas hydrate particles to produce fragmented gas hydrate particles; and
recycling into said reaction chamber at least a portion of said fragmented gas hydrate particles.

2. The method of claim 1, wherein the step of flowing water into said reaction chamber comprises atomizing said water.

3. The method of claim 1, wherein between the step of fragmenting at least a portion of material and recycling into said reaction chamber at least a portion of said fragmented gas hydrate particles, the method further comprises fluidizing said fragmented gas hydrate particles.

4. The method of claim 3, wherein the step of fluidizing said fragmented gas hydrate particles is carried in a second reaction chamber.

5. The method of claim 4, wherein the step of fluidizing is carried out with a hydrate forming gas.

6. The method of claim 1, further comprising recycling to said reaction chamber un-reacted hydrate forming gas.

7. The method of claim 6, wherein said un-reacted hydrate forming gas is compressed and cooled prior to the step of recycling un-reacted hydrate forming gas.

8. The method of claim 1, wherein the interior of said reaction chamber is held at a temperature between 255-320 K and a pressure ranging from 100-50,000 kPa.

9. The method of claim 8, wherein said temperature is controlled by adjusting the inlet temperature of said hydrate-forming gas and water streams with at least one refrigeration unit.

10. The method of claim 8, wherein said pressure is controlled by adjusting the inlet pressures of said gas, said water and solid inlet streams.

11. The method of claim 1, wherein said hydrate-forming gas is selected from the group consisting of methane, propane, ethane, carbon dioxide and natural gas.

12. The method of claim 1, further comprising optimizing the contact between said water, said gas and said hydrate particles.

13. The method of claim 12, wherein the step of optimizing the contact including adjusting the number, geometry, locations (above and/or in the bed), positions (angle of fluid jets) and operating conditions (fluid flow rates and pressures) of the atomizing devices.

14. The method of claim 1, wherein the step of fragmenting is carried out with a particle size reduction device selected from the group consisting of a crusher and a roll mill.

15. The method of claim 1, wherein the step of fragmenting can also be at least partially performed in-situ in said reaction chamber by fragmenting said growth particles with a particle size reduction device selected from the group consisting of a fluid jet from a water atomizing device and a feed gas nozzle.

16. The method of claim 1, wherein the step of providing seed gas hydrate particles in a reaction chamber comprises inserting seed material into said reaction chamber to assist in achieving steady state operation.

17. The method of claim 16, wherein said seed material is selected from the group consisting of seeds of ice particles and hydrate crystals.

18. The method of claim 1, wherein the step of providing seed gas hydrate particles in a reaction chamber comprises performing a start-up procedure, wherein said start-up procedure comprises:
setting the temperature of said reaction chamber at or below the freezing point of water;
producing ice in said reaction chamber by flowing atomized water having a diameter of less than 1 mm into said reaction chamber; and
flowing said hydrate-forming gas into said reaction chamber to produce said seed gas hydrate particles.
